(19) United States
(12) Patent Application Publication
(10) Pub. No.: US 2014/0057008 A1
(43) Pub. Date: Feb. 27, 2014

(54) METHODS OF PROCESSING EXTRACTS OF A T. WILFORDII HOOK F. PLANT

(71) Applicants: George Paffendorf, Hackettstown, NJ (US); Christopher M. Cimarusti, Manalapan, NJ (US)

(72) Inventors: George Paffendorf, Hackettstown, NJ (US); Christopher M. Cimarusti, Manalapan, NJ (US)

(21) Appl. No.: 14/002,529

(22) PCT Filed: Mar. 1, 2012
(86) PCT No.: PCT/US12/27231

§ 371 Date: Nov. 13, 2013

Related U.S. Application Data
(60) Provisional application No. 61/448,028, filed on Mar. 1, 2011.
(60) Provisional application No. 61/484,031, filed on May 9, 2011.

(51) Int. Cl. A61K 36/37

(52) U.S. Cl. CPC A61K 36/37
USPC 424/759

(57) ABSTRACT

Products derived from the Tripterygium wilfordii Hook F (TwHF) plant in the form of extracts are provided that are useful in preventing, treating, or ameliorating a symptom of an inflammatory disorder or an immune disorder, such as an auto-immune disease. Also provided are methods of freeze-drying a TwHF root and/or root portion, methods of debarking a freeze-dried TwHF root and/or root portion, methods of preparing and/or refining extracts in freely flowable solid form from freeze-dried debarked TwHF roots and/or root portions, and methods of using freely flowable solid extracts of TwHF in preventing, treating or ameliorating a symptom of an inflammatory disorder and/or an immune disorder.

### CROSS-REFERENCE TO RELATED APPLICATIONS

The present application claims the benefit of priority to U.S. Provisional Application No. 61/448,028, filed Mar. 1, 2011 and U.S. Provisional Application No. 61/484,031, filed May 9, 2011. The disclosure of each priority application is incorporated herein by reference in its entirety.

### FIELD OF THE INVENTION

The disclosed subject matter generally relates to plants and plant products and more specifically relates to Tripterygium wilfordii Hook F. plants, plant products and methods of making and using extracts of such plants and plant products.

### BACKGROUND OF THE INVENTION

Tripterygium wilfordii Hook F. (TwHF) (Celastraceae), commonly known as Thunder god vine, is a perennial shrub indigenous to China and Southeast Asia. TwHF contains a number of chemical compounds that are toxic. Parts of the TwHF plant, such as the leaves, the stem, flowers, and the skin and/or bark of the roots are poisonous and may cause death if ingested. TwHF has been used in traditional Chinese medicine to treat diseases and disorders, but the uses are limited by the significant toxicity risk. Administration of TwHF has also been shown to inhibit interleukin-2-mediated immunosuppression. Administration of a TwHF extract or its components, (i.e., triptolide and tripdiolide, compounds obtained from TwHF)), has been shown to inhibit interleukin-2 (IL-2). Purification of such compounds is costly and time-consuming, but necessary in view of the toxins. Use of less purified materials from the plant presents the risk that the benefits of therapy will be outweighed by the risks of deleterious consequences resulting from administration of toxic substances.

The inflammatory response is associated with invasion of a body by a foreign object or injury. On occasion, inflammatory responses, both acute and chronic, develop under circumstances where the body is not under true threat, and the inflammatory response itself becomes a condition or disorder requiring treatment. Analogously and closely aligned, the immune response wards off invasion by foreign objects, but that response also occurs under inappropriate circumstances, such as when there is no foreign object. A variety of disorders are characterized by inappropriate immune responses, exemplified by the various auto-immune disorders known to man. A continuing effort is being made to provide more effective and safer treatments for disorders involving inappropriate immune responses and/or inappropriate inflammatory responses.

For the foregoing reasons, a need continues to exist in the art for compositions comprising the beneficial biologically active compounds of TwHF with an acceptably reduced presence of TwHF toxins, that are useful in preventing, mitigating, treating or ameliorating a symptom of an inappropriate inflammatory response and/or an inappropriate immune response.

### SUMMARY OF THE INVENTION

Described herein are improved methods for obtaining and processing extracts of the Tripterygium wilfordii Hook F. (TwHF) plant that provide beneficial biologically active compounds with an acceptably reduced level of toxic substances that improves the safety profile. The methods yield natural products that provide the benefits of TwHF without undue risk of harming recipients that characterizes use of the native plant.

In one aspect, described herein is a method of producing freeze-dried root portions from roots of the TwHF plant, wherein the method comprises placing a plurality of frozen TwHF root portions in a container within a freeze-drying chamber, wherein the root portions are sufficiently close in proximity to each other within the container to facilitate heat transfer, and wherein the chamber is at a temperature of −30° C. or lower; lowering the air pressure within the chamber; and heating the container in an amount sufficient to sublimate the water in the root portions, thereby producing freeze-dried root portions of the TwHF plant. In one embodiment, the freeze-drying process is continued until the freeze-dried root portions contain no more than 20% (w/w) water. In another embodiment, the freeze-drying process is completed when the container temperature reaches a temperature of about 40° C. A freeze-dried root of a TwHF plant produced by a freeze-drying method described herein is also provided.

In some embodiments, the roots are harvested from a TwHF plant that has been cultivated for less than 30 months. In some embodiments, the roots are harvested from TwHF var. Shiloh. It is desirable in some embodiments to process the roots of the TwHF plant into root portions of a specific, average, or maximal length. In some of these embodiments, the roots are processed into root portions of a length of 6 inches or less prior to the start of the freeze-drying process. In some embodiments, the roots are processed to separate branched portions of a root into individual root portions, for example, substantially linear root portions of varying lengths.

In some embodiments, the container is placed upon a heated support within the freeze-drying chamber. The support may be directly heated, but the disclosure also comprehends that the heating of the container comprises incrementally increasing the temperature of the heated support. The container can be of any size, shape and depth. In some embodiments, the container has a depth of no more than 2 inches. In some embodiments, the heating of the container does not result in the heating of the freeze-drying chamber. In some embodiments, the container is a substantially planar shelf, e.g., a planar surface having a raised perimeter lip of 0.125, 0.25, 0.5, 1.0, 1.5, or 2 inches (height) or any dimensions within the range of 0.125-2 inches, or a nominal height.

In some embodiments, the root portions in the container are maintained at a temperature ranging from about 20° C. to about 70° C. In one embodiment, the root portions in the container are maintained at a temperature of about 38° C.

In another aspect, a method of producing freeze-dried debarked root portions (i.e., freeze-dried root cores) of a TwHF plant is also provided. In some embodiments, the method comprises separating the bark from the root core of a freeze-dried root portion, thereby producing a freeze-dried debarked root portion of a TwHF plant. In some embodiments, the separating step comprises applying mechanical force to the freeze-dried root portion in an amount effective to release the bark from the root core. In some embodiments, the method is optionally performed within an apparatus selected from the group consisting of a drum debarking device, a ring debarking device and a flail debarking device.

The mechanical force may be produced by any means known in the art. In some embodiments, the mechanical force is produced by contacting a freeze-dried root portion with an abrading agent selected from the group consisting of a freeze-dried root portion, a metal object, a glass object, a wooden object, a ceramic object, rope, leather, sand, rock, cement, concrete, garnet, aluminum oxide, silicon oxide, fiber glass, diamond, an internal projection of a debarking drum, an even or uneven internal surface of a debarking drum, an even or uneven internal surface of a ring debarking device, a plurality of strands of a flail, or a plurality of objects attached to a plurality of strands of a flail. In one embodiment, the abrading agent is a magnetized metal object.

In one embodiment, the method comprises use of an apparatus that is a drum debarking device and the abrading agent is a loose material present at a weight ratio of freeze-dried root portions to abrading agent of 1:8. In another embodiment, the abrading agent is a loose material present at a weight ratio of freeze-dried root portions to abrading agent of 1:2. In some embodiments, the freeze-dried root portions are tumbled with the abrading agent under vacuum for a time sufficient to release the bark from the freeze-dried root portion, wherein the released bark is removed by vacuum (e.g., through a port in the apparatus), thereby separating the bark from the root core.

Another aspect provides a freeze-dried debarked root portion of a TwHF plant produced by a method described herein. Some embodiments provide a freeze-dried root core of a TwHF plant, wherein the root core is substantially free of bark. In some embodiments, the root core contains no more than 5% (w/w) water. In some embodiments, the root core comprises less than 1% of the bark of a comparably dimensioned fresh root portion of a TwHF plant. In some embodiments, the lack of bark is determined by visual inspection for example, a lack of pink/light red powder on a debarked portion of a TwHF plant is indicative of a lack of bark.

In some embodiments according to this aspect of the disclosure, the root core comprises no more than 10% of a compound present in the bark of a TwHF plant. Such compounds (e.g., celastrol) can be used as markers to determine whether residual bark is present freeze-dried debarked root portions of T. wilfordii Hook F., or in extract preparations thereof. Thus, also described herein is a method of identifying residual bark in a preparation of a freeze-dried debarked root portion of a TwHF plant, wherein the method comprises determining the amount of a bark marker (e.g., celastrol) in the freeze-dried debarked root portion, wherein lower amounts of the marker (e.g., celastrol) in the freeze-dried debarked root portion preparation identifies the root portion preparation as containing lower amounts of residual bark. Those of skill in the art will recognize that a suitable threshold level for bark contamination of a root extract can vary depending on the nature of the extract and its intended use and, accordingly, the disclosure contemplates freeze-dried debarked root portions having no more than the threshold level of a bark marker such as celastrol, even if that threshold is greater than the measurable or detectable amount of the marker in the root portions. The amount of marker (e.g., celastrol) in the freeze-dried root portion preparation is determined by a method known in the art, such as NMR or HPLC.

In some embodiments, a preparation of a freeze-dried debarked root portion comprises less than 10% celastrol compared to the amount of celastrol present in bark of a comparably dimensioned fresh root of a TwHF plant. In some embodiments, the freeze-dried debarked root portion preparation comprises less than 5% celastrol compared to the amount of celastrol present in bark of a comparably dimensioned fresh root of a TwHF plant. In other embodiments, the freeze-dried debarked root portion preparation comprise less than 1% celastrol compared to the amount of celastrol present in bark of comparably dimensioned fresh root of a TwHF plant. In still other embodiments, the freeze-dried debarked root portion preparations comprise less than 0.9%, 0.8%, 0.7%, 0.6%, 0.5%, 0.4%, 0.3%, 0.2% 0.1% or less celastrol compared to the amount of celastrol present in bark of a comparably dimensioned fresh root portion of a TwHF plant.

Another aspect provided herein is an extract of a TwHF plant, optionally in the form of a freely flowable solid, that comprises no more than 2.5 μg of celastrol per mg of extract. In some embodiments, an extract of a TwHF plant having a ratio of (triptolide+tripdiolide)/celastrol of at least 1.36 is provided. In some embodiments, the ratio of (triptolide+tripdiolide)/celastrol content in the TwHF extract is at least 1.46.

Other aspects provided herein comprise methods of processing an extract, e.g., an alcoholic extract, of a TwHF plant into a freely flowable solid. One aspect provides a method that comprises obtaining an alcoholic extract of a TwHF plant, exchanging the alcohol in the alcoholic extract for a polar organic solvent such as ethyl acetate to form an extract/ethyl acetate mixture; combining the extract/ethyl acetate mixture with an excipient under vacuum at a temperature of no more than 50° C. to produce a concentrated mixture; and drying the concentrated mixture at a temperature of no more than 55° C., thereby producing the alcoholic extract in the form of a freely flowable solid. In some embodiments, the extract is obtained by one or more ethanol extractions from the roots of a TwHF plant. In some embodiments, the root is a freeze-dried debarked root of the TwHF plant. In other embodiments, the root is a fresh debarked root of the TwHF plant. Processing an extract of a TwHF plant by a method described herein can be performed on an extract prepared from any T. wilfordii root or root preparation, including an extract prepared from a debarked T. wilfordii root. Moreover, such roots can be debarked by any means known in the art, and thus need not be debarked by any particular process, such as a process involving a drum debarking device, a ring debarking device or a flail debarking device, as described in some embodiments herein. For example, extracts produced from roots of a TwHF plant that have been debarked through the use of compression rollers, peeling or other means of applying mechanical force are contemplated for practicing the extract processing methods described in the disclosure. In methods of freeze-drying T. wilfordii roots and root portions, in methods of preparing extracts from debarked roots and methods of processing existing T. wilfordii extracts, of course, no debarking process at all is needed or involved.

In another aspect, the method comprises mixing a native TwHF plant extract with an alcohol to produce an extract/alcohol mixture; combining the extract/alcohol mixture with an excipient under vacuum at a temperature of no more than 50° C. to produce a concentrated mixture; and drying the concentrated mixture at a temperature of no more than 55° C., thereby producing the extract in the form of a freely flowable solid. In some embodiments, the alcohol is ethanol. In some embodiments, the drying step is performed at a pressure of no more than 0.2 atmospheres (atm).

Excipients suitable for use in the methods described herein include an excipient selected from the group consisting of microcrystalline cellulose (MCC), maltodextrin, Aerosil® (fumed silica), corn starch, and Neusilin (magnesium aluminometasilicate). In some embodiments, the excipient is microcrystalline cellulose.

Yet another aspect described herein is a method of processing an alcoholic extract of a TwHF plant into a freely flowable solid, wherein the method comprises obtaining an alcoholic extract of a TwHF plant, exchanging the alcohol in the alcoholic extract for a polar organic solvent such as ethyl acetate to form an extract/ethyl acetate mixture; combining the extract with a non-polar organic solvent to produce an extract/non-polar organic solvent mixture; filtering the extract/non-polar organic solvent mixture to isolate a solid precipitate; and drying the solid precipitate, thereby producing the alcoholic extract in the form of a freely flowable solid. Non-polar organic solvents suitable for use in the method include a non-polar organic solvent selected from the group consisting of straight-chain alkanes (including, but not limited to, pentane, hexane, heptane, octane, nonane, and decane) and branched-chain alkanes (including, but not limited to, isopentane, isohexane, isoheptane, isooctane, isononane and isodecane). In some embodiments, the non-polar organic solvent is heptane.

In yet another aspect, described herein is a method of processing a native extract of a TwHF plant into a freely flowable solid, the method comprising combining the native extract with a non-polar organic solvent to produce a mixture thereof; filtering the mixture to isolate a solid precipitate; and drying the solid precipitate, thereby producing the native extract in the form of a freely flowable solid.

The disclosure also provides an extract of a TwHF plant in the form of a freely flowable solid produced by any of the aforementioned processing methods.

The following numbered paragraphs each succinctly define one or more exemplary variations of the invention.

1. An extract of a T. wilfordii Hook F. (TwHF) plant that comprises no more than 2.5 μg of celastrol per mg of the extract.

2. The extract of paragraph 1, wherein the extract has a ratio of (triptolide+tripdiolide)/celastrol ((T+Td)/C) of at least 1.46.

3. The extract of paragraph 2, wherein the extract has a ratio of (T+Td)/C of at least 1.36.

4. The extract of paragraph 1, wherein the extract is in the form of a freely flowable solid.

5. A method of producing a freeze-dried root portion from a root of a T. wilfordii Hook F. (TwHF) plant comprising:

(a) placing a frozen TwHF root portion in a container within a freeze-drying chamber, wherein the chamber is at a temperature of −30° C. or lower;

(b) lowering the pressure within the chamber; and

(c) heating the container sufficiently to sublimate the water in the root portion, thereby producing the freeze-dried root portion of the TwHF plant.

6. The method of paragraph 5, wherein a plurality of root portions are used and wherein the plurality of root portions are sufficiently close in proximity to each other within the container to facilitate heat transfer among the root portions.

7. The method of paragraph 5, wherein the root is harvested from a TwHF plant that has been cultivated for less than 30 months.

8. The method of paragraph 5, wherein prior to freeze-drying, the root is processed into a root portion length of 6 inches or less.

9. The method of paragraph 8, wherein the root is processed to be a substantially linear root portion.

10. The method of paragraph 5, wherein the container is placed upon a heated support within the freeze-drying chamber.

11. The method of paragraph 5, wherein the heating of the container does not involve direct heating of the freeze-drying chamber.

12. The method of paragraph 5, wherein the container is a substantially planar shelf.

13. The method of paragraph 5, wherein the root portion in the container is maintained at a temperature ranging from about 20° C. to about 70° C.

14. The method of paragraph 5, wherein the freeze-drying process is continued until the freeze-dried root portion contains no more than 20% (w/w) water.

15. The method of paragraph 5, wherein the root is harvested from TwHF var. Shiloh.

16. A freeze-dried root portion of a T. wilfordii Hook F. plant produced by the method of paragraph 5.

17. A method of producing a freeze-dried debarked root portion of a T. wilfordii hook F. (TwHF) plant comprising separating the bark from a root core of a freeze-dried root portion, thereby producing a freeze-dried debarked root portion of a TwHF plant.

18. The method of paragraph 17, wherein the separating step comprises applying sufficient mechanical force to a freeze-dried root portion of the TwHF plant to release the bark from the root core.

19. The method of paragraph 18, wherein the mechanical force is produced by contacting a freeze-dried root portion with an abrading agent selected from the group consisting of a freeze-dried root portion, a metal object, a glass object, a wooden object, a ceramic object, rope, leather, sand, rock, cement, concrete, garnet, aluminum oxide, silicon oxide, fiber glass, diamond, an internal projection of a debarking drum, an even or uneven internal surface of a debarking drum, an even or uneven internal surface of a ring debarking device, a plurality of strands of a flail, and a plurality of objects attached to a plurality of strands of a flail.

20. The method of paragraph 17, wherein the separating step comprises an apparatus selected from the group consisting of a drum debarking device, a ring debarking device and a flail debarking device.

21. A freeze-dried debarked root portion or freeze-dried root core of a TwHF plant produced by the method of paragraph 17.

22. A freeze-dried root core of a TwHF plant, wherein the root core is substantially free of bark.

23. The freeze-dried root core of paragraph 22, wherein the root core comprises less than 1% of the bark of a comparably dimensioned fresh root portion of a TwHF plant.

24. The freeze-dried root core of paragraph 22, wherein the root core comprises no more than 10% celastrol.

25. A method of processing a native extract of a TwHF plant to produce an extract in the form of a freely flowable solid, the method comprising

(a) mixing the native extract with an alcohol to produce a mixture thereof;

(b) combining the mixture with an excipient under vacuum at a temperature of no more than 50° C. to produce a concentrated mixture; and

(c) drying the concentrated mixture at a temperature of no more than 55° C. to remove remaining ethanol, thereby producing the extract in the form of a freely flowable solid.

26. The method of paragraph 25, wherein the extract is obtained by one or more ethanol extractions from the root of the TwHF plant.

27. The method of paragraph 25, wherein the native extract is obtained from a freeze-dried debarked root portion, or freeze-dried root core, of the TwHF plant.

28. The method of paragraph 25, wherein the excipient is selected from the group consisting of microcrystalline cellulose, maltodextrin, Aerosil® (fumed silica), corn starch, and Neusilin (magnesium aluminometasilicate).

29. The method according to paragraph 28, wherein the excipient is microcrystalline cellulose.

30. The method according to paragraph 25, wherein the drying step is performed under a vacuum establishing an air pressure of no more than 0.2 atm.

31. A method of processing an native extract of a TwHF plant to produce the extract in the form of a freely flowable solid, the method comprising:

(a) combining the native extract with a non-polar organic solvent to produce a mixture thereof;

(b) filtering the mixture to isolate a solid precipitate; and

(c) drying the solid precipitate, thereby producing the extract in the form of a freely flowable solid.

32. The method of paragraph 31, wherein the native extract is obtained by one or more ethanol extractions from the root of the TwHF plant.

33. The method of paragraph 31, wherein the non-polar organic solvent is selected from the group consisting of a straight-chain C5-C10 alkane and a branched-chain C5-C10 alkane.

34. The method of paragraph 33, wherein the non-polar organic solvent is heptane.

35. The method of paragraph 31, wherein the extract is obtained from a freeze-dried debarked root portion of a TwHF plant.

36. An extract of a TwHF plant in the form of a freely flowable solid produced by the method of paragraph 25.

37. The extract of paragraph 36, wherein the extract comprises no more than 2.5 μg of celastrol per mg of extract.

38. The extract of paragraph 36, wherein the extract has a ratio of (triptolide+tripdiolide)/celastrol ((T+Td)/C) of at least 1.46.

39. The extract of paragraph 36, wherein the extract has a ratio of (T+Td)/C of at least 1.36.

40. An extract of a TwHF plant in the form of a freely flowable solid produced by the method of paragraph 31.

41. The extract of paragraph 40, wherein the extract comprises no more than 2.5 μg of celastrol per mg of extract.

42. The extract of paragraph 40, wherein the extract has a ratio of (triptolide+tripdiolide)/celastrol ((T+Td)/C) of at least 1.46.

43. The extract of paragraph 40, wherein the extract has a ratio of (T+Td)/C of at least 1.36.

44. A method of processing an alcoholic extract of a TwHF plant to produce an extract in the form of a freely flowable solid comprising:

(a) obtaining an alcoholic extract of a TwHF plant;

(b) exchanging the alcohol in the alcoholic extract for a polar organic solvent to form an extract/polar organic solvent mixture;

(c) combining the extract/polar organic solvent mixture with an excipient to form an admixture;

(d) concentrating the admixture under vacuum at a temperature of no more than 50° C.; and

(e) drying the concentrated mixture at a temperature of no more than 55° C., thereby producing the extract in the form of a freely flowable solid.

45. The method of paragraph 44, wherein the polar organic solvent is ethyl acetate.

46. A method of processing an alcoholic extract of a TwHF plant to produce the extract in the form of a freely flowable solid comprising:

(a) obtaining an alcoholic extract of a TwHF plant;

(b) exchanging the alcohol in the alcoholic extract for a polar organic solvent to form an extract/polar organic solvent mixture;

(c) combining the alcoholic extract with a non-polar organic solvent to form an admixture;

(d) filtering the admixture to isolate a solid precipitate; and

(e) drying the solid precipitate, thereby producing the extract in the form of a freely flowable solid.

47. The method of paragraph 46, wherein the non-polar organic solvent is heptane.

Other features and advantages of the present invention will be better understood by reference to the following detailed description, including the Examples.

### DETAILED DESCRIPTION

The present application is based on the discovery that freeze-drying the roots of a TwHF plant facilitates comprehensive removal of the bark from the root core, which allows for the preparation of extracts that substantially lack the toxic compounds (e.g., celastrol) present in the bark.

The present application is also based on the discovery that non-polar organic solvents and formulation excipients are capable of transforming conventional extracts of a TwHF plant into a freely flowable solid. Conventional extracts of a TwHF plant produced by methods known in the art (e.g., alcoholic extractions of a TwHF plant without any further processing steps) are waxy, amorphous compositions (i.e., a form of native extract) with poor flow characteristics that require extraordinary means to manipulate and produce a useable drug product. The freely flowable solids produced by the processing methods described herein contain useful levels of extract compounds (e.g., triptolide and tripdiolide), and these extract compounds can be formulated into a drug product for the treatment of various disorders, including anti-inflammatory disorders such as rheumatoid arthritis.

### I. Definitions

Consistent with its meaning in the art, the term “extract” refers to material that is drawn out of a composition of matter, as by being physically drawn out, or pushed, by a fluid or by being at least partially dissolved in a solvent capable of contacting, and preferably penetrating, the composition of matter. Extract is also used to refer to the drawn out material as it exists upon extraction or following subsequent manipulation, e.g., to produce a solid. Accordingly, the extract may be in the form of a solution, a mixture, a concentrate or an essence. Solid forms of the extracts are substantially free, and typically free, of fluid used in extraction, e.g., a solvent. An extract is defined herein to include a single extract obtained from one or more extraction steps as well as an extract that is the combined product of a plurality or series of separate extractions, with each individual extraction comprising at least one extraction step.

The term “alcoholic extract of a TwHF plant” as used herein refers to material that is drawn out of a TwHF plant (e.g., a root portion of a TwHF plant) by one or more alcoholic extractions without being subjected to further processing steps including, for example, solvent exchange with a polar organic solvent (e.g., ethyl acetate) and/or isolation of solid particulates in the alcoholic extract. The alcoholic extract may be in the form of a solution, a mixture, a concentrate or an essence. Typically, the alcoholic extract is not a waxy, amorphous solid and does not contain a waxy, amorphous solid, but partial or complete removal of alcohol can produce an extract that is, or contains, a waxy, amorphous solid, referred to herein as one form of a native extract.

The term “native extract of a TwHF plant” as used herein includes an extract of the TwHF plant in the form of the waxy, amorphous solid that is produced by conventional methods known in the art. One form of the native extract, therefore, is an alcohol extract that is, or contains, a waxy, amorphous solid. Other alcoholic extracts of a TwHF plant are not necessarily extracts of native plants or plant parts and thus may be free of the waxy, amorphous solid.

The term “concentrated filtrate” as used herein is the result of concentrating one or more filtrates produced during an extraction process described herein.

The term “fresh root of a TwHF plant” as used herein refers to a root of a TwHF plant that has not been dried, e.g., freeze-dried.

The term “root portion” as used herein refers to part or all of a root of a TwHF plant that is of suitable size for placing into a freeze-drying chamber. To achieve this suitable size, a TwHF root may be processed or milled to a length (or diameter) compatible with freeze-drying in a freeze-drying chamber.

The term “abrading agent” as used herein refers to an agent that is capable of wearing down or eroding a material (e.g., bark on the roots of a TwHF plant) through friction and/or through mechanical impact.

The phrase “root portion substantially free of bark” is given its ordinary and accustomed meaning in the art of a root portion that generally lacks bark. In particular, it is contemplated that a root portion substantially free of bark comprises less than 2% of the bark (as determined by visual inspection) of a comparably dimensioned fresh root portion of a TwHF plant.

The terms “freely flowable solid” and “processed TwHF extract” are used interchangeably herein. In some embodiments, the native extract is combined with a non-polar organic solvent to produce a freely flowable solid extract suitable for formulation into drug products and/or nutritional supplements. Without wishing to be bound by theory, it is believed that the non-polar organic solvent (e.g., hexane, heptane) removes waxes, fats and oils present in the native extract that make it difficult to manipulate and formulate into deliverable forms (drug products or nutritional supplements). In some embodiments, the extract (e.g., an alcoholic extract that has been subjected to solvent exchange with a polar organic solvent such as ethyl acetate) is processed by contacting the extract with an excipient to form a freely flowable solid form of the extract amenable to manipulation and formulation into drug products and nutritional supplements.

### II. Methods of Freeze-Drying Roots of the TwHF Plant

As demonstrated in the Examples provided herein, freeze-drying the roots or root portions of a TwHF plant prior to removing the bark allows for easy, quantitative removal of the bark compared to conventional methods. Freeze-drying is a dehydration process in which water is removed from the root of a TwHF plant without exposing the plant material to protracted periods of exposure to the potentially deleterious effects of liquid water and/or excessive heat. The freeze-drying process can be performed with any method known in the art as well as in any freeze-drying apparatus known in the art.

One aspect of the disclosure provides a method of producing freeze-dried root portions from the roots of a TwHF plant, wherein the method comprises placing a plurality of frozen root portions in a container within a freeze-drying chamber, wherein the roots are sufficiently close in proximity to each other within the container to facilitate heat transfer; lowering the pressure within the chamber; and heating the container by an amount sufficient to sublimate the water in the root portions, thereby producing freeze-dried root portions of a TwHF plant.

The first step of the freeze-drying process is the freezing of the root portions. The roots of the TwHF plant can grow to a length of up to 12 meters, which is too large to fit into most commercial freeze-drying chambers. Thus, in some embodiments, it is desirable to process or mill the roots of the TwHF plant prior to beginning the freeze-drying process in order to produce root portions of a more manageable size. The TwHF roots are processed to a desired length or range of lengths before or after freezing, but prior to freeze-drying. For example, in some embodiments, the roots of the TwHF plant are processed to produce root portions of a length of about 24 inches or less. In some embodiments, the roots of the TwHF plant are processed to produce root portions of a length of about 12 inches, about 11 inches, about 10 inches, about 9 inches, about 8 inches, about 7 inches, about 5 inches, about 4 inches, about 3 inches, about 2 inches, about 1 inch, about ½ inch, or less. In some embodiments, the roots of the TwHF plant are processed to produce root portions of a length of about 6 inches.

In some embodiments, a root of the TwHF plant has a diameter of about ½ inch or less. In some embodiments, a root has a diameter of about ⅓ inch, ¼ inch, ⅛ inch, 1/16 inch or less. In other embodiments, a root of the TwHF plant has a diameter of about 1 inch or more. In one embodiment, the roots of the TwHF plant are processed to produce root portions of a length of about 6 inches and a diameter of about ½ inch. Typically, TwHF roots are not processed to alter the average diameter thereof, but the disclosure contemplates such processing using any conventional milling technique where reduced diameters and increased surface are desired (or at least not undesired) to more efficiently freeze-dry the preparation. Given that the roots or root portions are subject to extraction, various lengths and diameters of roots and root portions are acceptable, including ground, macerated or pulverized roots and root portions.

In some embodiments, the roots of the of the TwHF plant are branched. In some embodiments, the branched roots of the TwHF plant are processed to separate the branched sections of the roots to produce substantially linear root portions.

The root portions to be used in a freeze-drying process described herein are obtained from a TwHF plant of any age and origin. In some embodiments, the root portions are obtained from a Chinese-derived TwHF plant, and in some embodiments, the root portions are obtained from a United States-derived TwHF plant. In some embodiments, the root portions are obtained from TwHF var. Shiloh. The term “Chinese-derived TwHF plant” as used herein refers to a TwHF plant that is obtained from a Chinese source. This definition includes root portions harvested from a plant that is cultivated in China, roots portions harvested in the wild from a plant grown in China, and root portions harvested from a plant that is cultivated in a country other than China, but the plant is of Chinese origin (i.e., grown from a cutting (or a seed) of a Chinese TwHF plant). The term “United States-derived TwHF plant” as used herein refers to a TwHF plant that is obtained from a United States source. This definition includes root portions harvested from a plant that is cultivated in the United States, roots portions harvested in the wild from a plant grown in the United States, and root portions harvested from a plant that is cultivated in a country other than the United States, but the plant is of United States origin (i.e., grown from a cutting (or a seed) of a United States TwHF plant).

Root portions are obtained from a TwHF plant of any age. In some embodiments, the root portions are obtained from a TwHF plant that is grown for less than about 30 months. In related embodiments, the root portions are obtained from a TwHF plant that is cultivated for about 29 months, 28 months, 27 months, 26 months, 25 months, 24 months, 23 months, 22 months, 21 months, 20 months, 19 months, 18 months, 17 months, 16 months, 15 months, 14 months 13 months, 12 months, 11 months, 10 months, 9 months, 8 months, 6 months or less. In other embodiments, the root portions are obtained from a TwHF plant that is grown for at least 30 months or longer. In such embodiments, the root portions are obtained from a TwHF plant that is cultivated for about 31 months, 32 months, 33 months, 34, months, 35 months, 36 months, 37 months, 38 months, 39 months, 40 months, 41 months, 42 months, 43 months, 44 months, 45 months, 46 months, 47 months, 2 years, 3 years, 4 years, 5 years, 6 years, 7 years, 8 years, 9 years, 10 years or more.

A variety of approaches are suitable for freezing the TwHF roots or root portions from which extracts are to be prepared. In some embodiments, the freezing process is performed by placing the root portions in a container and placing the container in a dedicated freezing apparatus (i.e., a freezing apparatus that is separate from the freeze-drying chamber) overnight prior to beginning the freeze-drying process. The root portions are placed sufficiently close to each other within the container to facilitate heat transfer during the freeze-drying process. Preferably a root portion is in contact with at least one other root portion and/or with the container to facilitate heat transfer by conduction. The container may be composed of any material or materials, preferably conductive material(s). The container is of any shape or footprint compatible with the freeze-drying chamber. The container is any depth compatible with the internal dimensions of the freeze-drying chamber; in some embodiments, the container has a depth of no more than 2 inches, such as a depth in the range of about 0.125 to about 2 inches, or a nominal height. In some embodiments, a root portion is cooled below its eutectic point to ensure the absence of liquid-phase material, regardless of the pressure.

The use of any commercial freezing apparatus (e.g., freezer) that is capable of reducing the temperature of the root portions to a temperature ranging from −10° C. to −70° C. is specifically contemplated. In some embodiments, the temperature of the root portions is reduced to a temperature ranging from −20° C. to about −70° C. In some embodiments, the temperature of the root portions is reduced to a temperature of about −20° C., −25° C., −30° C., −35° C., −40° C., −45° C., −50° C., −55° C., −60° C., −65° C. or about −70° C. In some embodiments, the temperature of the root portions is reduced to a temperature of about −20° C. Once the roots have been frozen at the desired temperature, the container comprising the frozen root portions is placed in a freeze-drying chamber, the temperature of which is maintained at a temperature of about −20° C. or lower.

In other embodiments, the freezing process is performed within the freeze-drying chamber without the use of a dedicated, distinct freezing apparatus. In such embodiments, the root portions are placed directly into the freeze-drying chamber and the temperature of the chamber is reduced to about −20° C. or lower.

Once the freeze-drying chamber has reached the desired temperature and the root portions have been placed in the freeze-drying chamber, the air pressure in the chamber is lowered in order for water within the root portions to sublimate. In some embodiments, heat is applied to the root portions to facilitate removal of water from the root portions. During this process, at least 50% of the water in the root portions is sublimated. In some embodiments, at least about 60%, 65%, 70%, 75%, 80%, 85%, 90%, 95%, 96&, 97%, 98%, 99% or more of the water in the root portions is sublimated. In some embodiments, heat is applied to the root portions indirectly by incrementally increasing the temperature of the freeze-drying chamber from about 10° C. to about 70° C., resulting radiant heating as well as some conduction heating of the root portions. In some embodiments, the temperature of the chamber is first increased to about 10° C. and the temperature is stabilized at about 10° C. overnight. The temperature of the chamber is then increased to about 35° C. for about 8 hours and then increased to about 50° C. for about 8 hours. At the end of the freeze-drying process, the final residual water content in the freeze-dried root portions is, in some embodiments, about 20% or lower. In some embodiments, the freeze-dried root portions contain no more than 15% water. In other embodiments, the freeze-dried root portions contain no more than 10% water. In other embodiments, the freeze-dried root portions contain no more than 5% water or less. In other embodiments, the freeze-dried root portions contain no more than 2% water or less.

In other embodiments, the heat is applied directly to the root portions by incrementally increasing the temperature of a support within the freeze-drying chamber from about 10° C. to about 70° C., resulting in primarily, if not exclusively, conduction heating of the root portions. In some of these embodiments, frozen root portions are placed within a heat-conducting container upon a heated support within the freeze-drying chamber and the temperature of the heated support is increased incrementally. While the temperature of the freeze-drying chamber may increase as a byproduct of the heat supplied to the support within the chamber, the freeze-drying chamber itself is not being directly heated. In some embodiments, the temperature of the heated support is increased to about 10° C. overnight and then increased (from about 20° C. to about 40° C.) again for about 6-12 hours and increased again (from about 30° to about 60° C. for another 6-12 hours. The temperature of the root portions within the chamber is maintained at a temperature of about 38° C. In some embodiments, the heated support is heated intermittently, thereby providing intermittent heat to the root portions. In such embodiments, heat is typically provided to the root portions in 2- to 3-second intervals. In some embodiments the heated support provides intermittent heat to the root portions in 5-second, 10-second, 15-second, 20-second, 25-second, 30-second, 35-second, 40-second, 45-second, 50-second, 55-second, 1-minute, 5-minute, 10-minute, 15-minute, 20-minute, 25-minute, 30-minute, 35-minute, 40-minute, 45-minute, 50-minute, 55-minute, 1-hour, 2-hour, 3-hour, 4-hour, 5-hour, 6-hour, 7-hour, 8-hour, 9-hour, 10-hour intervals or more. Although the freeze-drying chamber is typically not heated in the embodiments described immediately above, it is contemplated that TwHF root portions may be freeze-dried in a freeze-drying chamber that is heated in addition to heating a support for the container with the root portions. At the end of the freeze drying process, the final residual water content in the freeze-dried root portions is, in some embodiments, about 20% or lower. In some embodiments, the freeze-dried root portions contain no more than 15% water, no more than 10% water, no more than 5% water or less.

The pressure of the freeze-drying chamber is controlled through the application of a vacuum. The vacuum is employed to facilitate sublimation. In some embodiments, the pressure within the freeze-drying chamber ranges from about 3 to about 10 millibar. In some embodiments, the pressure within the freeze-drying chamber is no more than 0.01 atm.

A cold condenser chamber and/or a condenser plate is/are used, in some embodiments, to provide a surface upon which water vapor may be contained and/or collected. By utilizing a condenser, water vapor is prevented from reaching the vacuum pump, which could degrade the performance of the pump. The condenser is maintained at a temperature known in the art as useful in de-humidification. In some embodiments, the temperature of the condenser is maintained at −50° C. or lower. In some embodiments, the temperature of the condenser is maintained at −30° C. or lower.

The resulting freeze-dried root portion of the TwHF plant comprises a root core and bark, wherein the attachment of the bark to the root core is altered, thereby reducing the strength of attachment of the bark to the root core relative to a fresh TwHF root portion of comparable dimensions. Without wishing to be bound to any particular theory, it is contemplated that a thoroughly dried root portion of a TwHF plant prepared, for example, according to a freeze-drying method described herein, allows for more quantitative removal of bark from the root core. Extracts obtained from such freeze-dried debarked root cores are expected to contain considerably lower levels of toxic compounds (e.g., celastrol) compared to extracts obtained from root cores in which the bark has been removed by conventional methods without first being freeze-dried or obtained from root cores that are freeze-dried, but wherein the freeze-drying process did not result in substantial sublimation of the water within the root portions.

### III. Methods of Debarking the Roots of the TwHF Plant

The aspect of the disclosure drawn to methods of debarking TwHF roots is distinct from such other aspects of the disclosure as methods of freeze-drying TwHF roots, methods of producing TwHF extracts, and methods of processing such extracts into a freely flowable solid. Nonetheless, these aspects of the disclosure are modular and are contemplated in various combinations, such as a method of producing an extract comprising a freeze-drying method, a debarking method, and an extract production method.

Accordingly, another aspect of the disclosure is a method for removing the bark from the roots of a TwHF plant that is less labor-intensive than peeling the bark from the roots or cutting the bark from the roots with a debarking blade such as a machete, a knife or a vegetable peeler. These known methods are laborious and are not suited for commercial manufacturing. The methods described herein are suitable for translation to a commercial manufacturing environment and comprise separating the bark from the root core of a freeze-dried root portion, thereby producing a freeze-dried debarked root portion of a TwHF plant Although the debarking methods are contemplated as useful in debarking a TwHF plant part (e.g., root), the debarking methods disclosed herein are also contemplated as useful in debarking a plant part (e.g., root or stem) of a variety of gymnosperm and angiosperm plants having a bark that is desirably removed from the plant or plant portion.

The bark of a freeze-dried root portion is separated from the root core by any means known in the art. For example, in some embodiments, separating the bark from the root core comprises mechanical disruption of the adhesion of the freeze-dried bark to the root core by mechanical force. In some embodiments, the mechanical force is produced by contacting the freeze-dried root portion with a compression roller or other means of compressing the root portion. The mechanical force is produced, in some embodiments, by contacting the freeze-dried root portion with an abrading agent. Exemplary abrading agents include, but are not limited to, a freeze-dried root portion, a metal object, a glass object, a wooden object, a ceramic object, rope, leather, sand, rock, cement, concrete, garnet, fiber glass, aluminum oxide, silicon oxide, diamond, and any other abrading agent known in the art. In some embodiments, the abrading agent is a metal object such as a bolt, screw, nail or metal fragment. Such metal objects can be obtained from any hardware store. In one embodiment, the abrading agent is a bolt. In some embodiments, the bolt is at least 3 inches in length. In other embodiments, the bolt is less than 1 inch in length, such as a bolt that is a ½ inch or less in length. In one embodiment, the bolt is a ½ inch in length, with a ⅜ inch head.

The method of producing freeze-dried debarked roots can be performed in any suitable apparatus known in the art. In some embodiments, the apparatus is any suitably sized device comprising compression rollers, preferably wherein the amount of pressure applicable to the root portion is adjustable. In other embodiments, the apparatus is a device selected from the group consisting of a drum debarking device, a ring debarking device and a flail debarking device. In certain embodiments, the apparatus is a drum debarking device and the abrading agent is a loose material present at a weight ratio of abrading agent to freeze-dried root portions of 12:1. In some embodiments, the weight ratio of abrading agent to freeze-dried root portions is 11:1, 10:1, 9:1, 8:1, 7:1, 6:1, 5:1, 4:1, 3:1, 2:1 or 1:1. The weight ratio of abrading agent to freeze-dried root portions is dependent upon the size of the root portions and the density of the abrading agent and can be determined by one of skill in the art. In some embodiments, the abrading agent is not a separate agent, but rather an internal projection of a debarking drum, an even or uneven internal surface of a debarking drum, an even or uneven internal surface of a ring debarking device, a plurality of strands of a flail, or a plurality of objects attached to a plurality of strands of a flail.

In some embodiments, the separating step comprises commingling the freeze-dried root portions with the abrading agent under vacuum for a time sufficient to release the bark from the freeze-dried root portion, wherein the released bark is removed through a port by vacuum, thereby separating the bark from the root core. In some embodiments, the freeze-dried root portions are tumbled with an abrading agent for at least 5 minutes, 10 minutes, 15 minutes, 20 minutes, 25 minutes, 30 minutes, 35 minutes, 40 minutes, 45 minutes, 50 minutes, 55 minutes, 1 hour, 2, 3, 4, 5, 6, 7, 8, 9, 10, 11, 12 hours or more.

Regardless of the apparatus or device used to remove the bark from the root portion, the debarking process is completed when the absence of bark on the root portions is apparent from visual inspection. Optionally, the duration of the debarking process is determined with an assay for a marker of TwHF root bark, such as celastrol, as described below.

In some embodiments, the method further comprises separating the abrading agent from the debarked root portions. For separation of the abrading agent from the debarked root portions, it is desirable, in some embodiments, to utilize a magnetizable (e.g., ferromagnetic) metal object as the abrading agent. In such embodiments, once the debarking process is completed, the metal object is magnetically removed utilizing a magnet that is either inside or outside of the apparatus. In some embodiments, once the debarking process is completed, the tumbler is magnetized and the magnetizable metal objects remain in the tumbler while the debarked root portions are removed from the tumbler.

Also provided herein is a freeze-dried debarked root portion of the TwHF plant, such as a T. wilfordii Hook F. var. Shiloh plant, wherein the root portion is substantially free of bark. In some embodiments, the freeze-dried debarked root portion of the TwHF plant comprises less than 2% or less than 1% of the bark of a comparably dimensioned fresh root portion of a TwHF plant. The presence of bark can be determined visually or through the use of a bark marker as described below. In some embodiments, the freeze-dried root portion comprises less than 1% of the bark of a comparably dimensioned fresh root portion of a TwHF plant. In some embodiments, the freeze-dried debarked root portion comprises less than 0.9%, 0.8%, 0.7%, 0.6%, 0.5%, 0.4%, 0.3%, 0.2% 0.1% or less of the bark of a comparably dimensioned fresh root portion of a TwHF plant.

Compounds present in the bark of a TwHF plant can be used as markers to determine whether a measurable amount of bark is present on the freeze-dried debarked root portions. Celastrol is a triterpenoid antioxidant compound that is present in the bark of a TwHF plant. Thus, in some embodiments, a method of identifying residual bark in a preparation of freeze-dried debarked root portions of a TwHF plant comprises determining the amount of a bark marker (e.g., celastrol) in the preparation of freeze-dried debarked root portions, wherein lower amounts of the marker in the preparation of freeze-dried debarked root portions identifies the preparation as containing lower amounts of residual bark. The amount of celastrol in the freeze-dried root portions can be determined by methods known in the art, such as NMR, HPLC or an Annexin V/PT staining assay (celastrol is an NF-κβ inhibitor).

In some embodiments, the freeze-dried debarked root portions comprise less than 10% celastrol compared to the amount of celastrol present in bark of a comparably dimensioned fresh root portions of a TwHF plant. In some embodiments, the freeze-dried debarked root portions comprise less than 5%, 4%, 3%, 2%, 1%, 0.9%, 0.8%, 0.7%, 0.6%, 0.5%, 0.4%, 0.3%, 0.2% 0.1% or less celastrol compared to the amount of celastrol present in bark of comparably dimensioned fresh roots of a TwHF plant. In some embodiments, if the process of removing the bark included the use of a drum and abrading agent and residual bark has been identified on the root portion the tumbling process is repeated until celastrol is undetectable.

### IV. Methods of Producing an Extract of a TwHF Plant

Methods of producing an extract from the roots of a TwHF plant are known in the art. Conventional extraction methods comprise grinding, milling or pulverizing plant material (e.g., debarked roots of a TwHF plant); extracting the plant material in a solution containing a sufficient amount of extractant fluid (e.g., an alcohol); and collecting the fluid to obtain an extract mixture (e.g., an alcoholic extract). The extract mixture may be further processed by removing solid matter from the extract mixture. Solid matter is removed from the extract by any method known in the art including, but not limited to, filtration and centrifugation. In some embodiments, the roots or root portions of the TwHF plant are debarked using the freeze-drying and debarking methods described herein. In other embodiments, the roots or root portions of the TwHF plant are fresh roots or portions that have been debarked using methods known in the art.

Grinding, milling or pulverizing debarked roots of a TwHF plant can be performed by any conventional milling process to increase the surface area of the root cores exposed to an extractant fluid.

In some embodiments, the extractant fluid for use in the extraction method is a solvent. Suitable extractant fluids include, but are not limited to, water, alcohols, aqueous solutions, halocarbons, esters and supercritical fluids. Suitable alcohols include primary alcohols such as ethanol, N-propanol, N-butanol, N-pentanol, N-hexanol, N-octanol, N-nonanol and N-decanol as well as secondary alcohols such as isopropanol, isobutanol, and the secondary alcohol derivatives of, e.g., any of butane through decane. For those lower molecular weight compounds that are gases at room temperature and about one atmosphere (atm), pressurized extractions in which the compounds are in a liquid state are contemplated. In one embodiment, the extractant fluid is ethanol. A benefit of incorporating an ethanolic fluid during the extraction process is that an ethanolic fluid is compatible with an ingestible product, and therefore is suitable for use in preparing an extract for incorporation into a pill, capsule, tablet, and other ingestible forms known in the art.

In one aspect, the extracting step comprises combining debarked roots, milled if desired, with an excess of extractant fluid such as 2- to 20-volumes of extractant fluid per unit volume of milled roots, and the combined materials are stirred for a time sufficient to extract the compounds of interest from the milled roots. In some embodiments, the mixture comprising the extractant fluid and the milled roots is stirred for about 30 minutes, 1 hour, 2 hours, 3 hours, 4 hours, 5 hours, 6 hours, 7 hours, 8 hours, 9 hours, 10 hours or longer. Optionally, the mixture is stirred at reflux. The extraction process is conducted at a temperature of between room temperature and the boiling point of the extractant fluid.

In some embodiments, the mixture is filtered and the filtrate is concentrated until the concentrated filtrate is a fraction of the volume of the initial filtrate. In some embodiments, the concentrated filtrate is less than 1, 0.9, 0.8, 0.7, 0.6, 0.5, 0.4, 0.3, 0.2, 0.1 or fewer volumes relative to the initial filtrate. In some embodiments, the roots, milled if desired, are subjected to a series of extractions with the extractant fluid. In such embodiments, the roots are again combined with an excess of extractant fluid and the extraction process is repeated. The filtrates obtained from the various extractions are then combined and concentrated as described below.

Concentration of the filtrate(s) is accomplished by methods known in the art, such as through the use of a vacuum to remove a volatile fluid, e.g., solvent. The concentration of the filtrate occurs at any temperature. In some embodiments, the temperature does not exceed 50° C. Concentrating the filtrate at a temperature of about 20° C., 25° C., 30° C., 35° C., 40° C., 45° C., 55° C., 60° C., 65° C., or 70° C. is also contemplated.

In some embodiments, solvent exchange is included in the extraction (U.S. Pat. Nos. 5,294,443; 5,500,340; 5,580,562; 5,846,742; 5,916,564, the disclosures of which are incorporated herein by reference in their entireties). In such embodiments, the extracting step comprises combining the concentrated filtrate with an excess (v/v) of a polar organic solvent (e.g., ethyl acetate) relative to original root mass to produce an extract mixture, which also may be concentrated as described above. The polar organic solvent (e.g., ethyl acetate) is different from the alcohol solvent used to prepare the extract in the extracting step. This process is repeated until the solvent exchange reduces the extractant fluid concentration in the extract mixture to 15% or less by weight. In some embodiments, the process is repeated until the extractant fluid concentration in the extract mixture is 10%, 9%, 8%, 7%, 6%, 5%, 4%, 3%, 2%, 1% or less by weight. The concentration of the extractant fluid can be determined by methods known in the art (e.g., NMR).

In some embodiments, the extract mixture is filtered to remove insoluble components, and the filtered extract solution is processed further by one of the methods described herein.

V. Methods of Processing an Alcoholic Extract of a TwHF Plant into a Freely Flowable Solid

In another aspect, described herein is a method of processing an alcoholic extract of a TwHF plant into a freely flowable solid. In some embodiments, the method comprises obtaining an alcoholic extract of a TwHF plant, combining the alcoholic extract with a non-polar organic solvent to produce an extract/non-polar organic solvent mixture; filtering the extract/non-polar organic solvent mixture to isolate a solid precipitate and drying the solid precipitate, thereby processing the alcoholic extract into the form of a freely flowable solid. In some embodiments, the alcoholic extract is a mixture subjected to solvent exchange prior to the combining step, wherein the solvent exchange comprises exchanging the alcohol in the alcoholic extract for a polar organic solvent (e.g., such as ethyl acetate). In such embodiments, the extract/ethyl acetate mixture is combined with the non-polar organic solvent in the combining step.

Suitable non-polar organic solvents include any of the C4-C10 straight- or branched-chain alkanes and cycloalkanes. In some embodiments, the non-polar organic solvent is a straight-chain alkane such as pentane, hexane, heptane, octane, nonane or decane. In some embodiments, the non-polar organic solvent is heptane. In other embodiments, the non-polar organic solvent is a branched-chain alkane, such as isopentane, isohexane, isoheptane, isooctane, isononane or isodecane. In some embodiments, the non-polar organic solvent is isooctane.

The combining step comprises contacting, mixing or putting together the alcoholic extract (or extract/ethyl acetate mixture) with an excess volume, such as 2- to 20-volumes, of a non-polar organic solvent relative to the original root mass to produce an admixture. In some embodiments, the combining step occurs under vacuum. In some embodiments, the combining step is repeated until the level of non-polar organic solvent in the admixture is undetectable. The admixture is filtered to isolate or collect solid precipitate material and subjected to one or more washes with the non-polar organic solvent. Filtering the admixture can be performed by conventional methods known in the art and can be repeated, if desired

In an alternative aspect, the method comprises combining the extract with an excipient under vacuum to produce an extract/excipient mixture; and drying the concentrated mixture, thereby producing the extract in the form of a freely flowable solid. The combining step comprises contacting, mixing and/or putting together the extract with an appropriate amount of excipient (compared to the total solid content in the extract), such as 0.5- to 20-fold (w/w), and optionally stirring for a time sufficient to mix the excipient and the extract. Use of an excipient in an amount of about 1-, 2-, 3-, 4-, 5-, 6-, 7-, 8-, 9-, 10-, 11-, 12-, 13-, 14-, 15-, 16-, 17-, 18-, 19-fold excess (w/w) relative to the solid content of the extract is also contemplated. Suitable excipients for use in the methods described herein include microcrystalline cellulose (MCC), maltodextrin, Aerosil® (fumed silica), corn starch, and Neusilin (magnesium aluminometasilicate). In some embodiments, the excipient is MCC. In some embodiments, the combining step comprises concentrating the extract/excipient mixture under full vacuum at a temperature not to exceed 50° C. (e.g., about 20° C., 30° C., 35° C., 36° C., 37° C., 38°, 39° C., 40° C., 41° C., 42° C., 43° C., 44° C., 45° C., 46° C., 47° C., 48° C. or about 49° C.) until a dry solids content of approximately 80% is achieved in the concentrated excipient mixture.

The drying steps of the processing methods described above comprise drying the washed solids, or concentrated excipient mixture, for a time sufficient to remove the solvents from the washed solids or concentrated excipient mixture. In some embodiments, the drying step is performed for a time period of about 8-24 hours. Drying the washed solids, or concentrated excipient mixture, for about 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours or more is specifically contemplated. Drying the washed solids, or concentrated excipient mixture, for less than 8 hours is also contemplated. The washed solids, or concentrated excipient mixture, can be dried in any drying apparatus known in the art including, but not limited to, a tray dryer, a vacuum oven or an oven. In some embodiments, the washed solids (or concentrated excipient mixture) are dried at a temperature of about 20° C., 25° C., 30° C., 35° C., 40°, 45° C. or about 50° C. After the drying process is completed, the dried solids are in the form of a freely flowable solid.

Extracts of TwHF contain more than 200 compounds, including diterpenoids, triterpenoids, sesquiterpenoids, β-sitosterol, dulcitol and glycosides. Exemplary compounds include, but are not limited to, triptolide, tripdiolide, polpunonic acid (wilfortrine) and the methyl ester thereof, triptophenolide, triptophenolide methyl ether, triptonoterpenol, wilformine, wilforine, wilforgine, and wilforzine. In some embodiments, it is desirable to determine the amount of one or more of these compounds in the freely flowable solid. The determination of compounds in a sample can be determined by methods known in the art such as NMR or HPLC.

The freely flowable solid produced by a method described herein contains concentrated levels of triptolide and tripdiolide. In some embodiments, the freely flowable solid produced by a method described herein comprises triptolide at a concentration of at least 100 μg, at least 200 μg, at least 300 μg, at least 400 μg, at least 500 μg, 600 μg, 700 μg, 800 μg, 900 μg, 1000 μg, 1100 μg, 1200 μg, 1300 μg, 1400 μg, 1500 μg, 1600 μg, 1700 μg, 1800 μg, 2000 μg or more per gram of the freely flowable solid. In some embodiments, the freely flowable solid comprises tripdiolide at a concentration of at 500 μg, 600 μg, 700 μg, 800 μg, 900 μg, 1000 μg, 1100 μg, 1200 μg, 1300 μg, 1400 μg, 1500 μg, 1600 μg, 1700 μg, 1800 μg, 2000 μg or more per gram of the freely flowable solid.

The freely flowable solid produced by a method described herein (using a freeze-dried debarked root portion, or freeze-dried debarked root core, as the starting material in the extraction process) contains low levels of bark compounds such as celastrol. Thus, the freely flowable solid has low levels of any bark toxins. In some embodiments, the freely flowable solid produced by a method described herein comprises no more than 2.5 μg of celastrol per mg of the freely flowable solid.

In some embodiments, the freely flowable solid has a ratio of (total weight of triptolide+tripdiolide)/(total weight celastrol) of 1 or more. In some embodiments, the ratio is more than 1.3 (e.g., 1.35, 1.36, 1.37, 1.38, 1.39, 1.4, 1.41, 1.42, 1.43, 1.44, 1.45, 1.46, 1.47, 1.48, 1.49, 1.5, 1.6, 1.7, 1.8, 1.9, 2, 2.5, 3, 3.5, 4, 4.5, 5, 5.5, 6, 6.5, 7, 7.5, 8, 8.5, 9, 9.5, 10 or more). In some embodiments, the weight ratio of (triptolide+tripdiolide)/celastrol is at least 1.46. In some embodiments, the weight ratio of (triptolide+tripdiolide)/celastrol is at least 1.36.

VI. Methods of Processing a Native Extract of a TwHF Plant into a Freely Flowable Solid

The native extract of a TwHF plant is a waxy, amorphous solid that requires extraordinary means to produce a useable drug product. Described herein are improved methods of processing the native extract of a TwHF plant into a freely flowable solid (e.g., a flowable powder).

In one aspect, the method comprises combining the native extract with a non-polar organic solvent to produce a mixture thereof; filtering the mixture to isolate or collect a solid precipitate and drying the solid precipitate, thereby producing an extract in the form of a freely flowable solid. Suitable non-polar organic solvents include any of the C4-C10 straight- or branched-chain alkanes and cycloalkanes. In some embodiments, the non-polar organic solvent is a straight-chain alkane such as pentane, hexane, heptane, octane, nonane or decane. In some embodiments, the non-polar organic solvent is heptane. In other embodiments, the non-polar organic solvent is a branched-chain alkane, such as isopentane, isohexane, isoheptane, isooctane, isononane or isodecane. In some embodiments, the non-polar organic solvent is isooctane.

In some embodiments, the non-polar organic solvent/extract mixture is filtered to isolate or collect solid precipitate material. Filtering the non-polar organic solvent/extract mixture can be performed by conventional methods known in the art. Once collected, the solids are subjected to one or more washes with a non-polar organic solvent.

In another aspect, the method comprises mixing a native extract of a TwHF plant with a polar organic solvent (e.g., an alcohol) to produce an extract/polar organic solvent mixture; combining the extract/polar organic solvent mixture with an excipient under vacuum at a temperature of no more than 50° C. to produce a concentrated mixture; and drying the concentrated mixture at a temperature suitable to remove remaining polar solvent from the mixture, thereby producing the native extract in the form of a freely flowable solid. Suitable polar organic solvents for use in this processing method include, but are not limited to, ethanol, ethyl acetate, isopropanol, n-butanol, n-propanol and methanol. In some embodiments, the polar organic solvent is ethanol.

In some embodiments, the adding step comprises incorporating an excess amount of an excipient (w/v), such as 0.5- to 20-fold, into the polar solvent/extract mixture and optionally stirring for a time sufficient to mix the excipient into the polar solvent/extract mixture to produce an excipient mixture. Mixing an excipient amount of about 1-, 2-, 3-, 4-, 5-, 6-, 7-, 8-, 9-, 10-, 11-, 12-, 13-, 14-, 15-, 16-, 17-, 18-, 19-fold excess (w/v) over the polar solvent/extract mixture is also contemplated. Suitable excipients for use in the methods described herein include microcrystalline cellulose (MCC), maltodextrin, Aerosil® (fumed silica), corn starch, and Neusilin (magnesium aluminometasilicate). In some embodiments, the excipient is MCC.

In embodiments where an excipient is added to the organic solvent/extract mixture, no filtration step is performed.

The washed solids, or excipient mixture, are then dried for a time sufficient to remove the organic solvent from the solids, or excipient mixture, respectively. In some embodiments, the drying step is performed for a time period of about 8-24 hours. Drying the washed solids, or excipient mixture, for about 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours or more is specifically contemplated. Drying the washed solids, or excipient mixture, for less than 8 hours is also contemplated. The washed solids, or excipient mixture, can be dried in the air or in any drying apparatus known in the art including, but not limited to, a tray dryer, a vacuum oven or an oven. In some embodiments, the washed solids, or excipient mixture, are dried in a drying apparatus at a temperature of about 20° C., 25° C., 30° C., 35° C., 40°, 45° C. or about 50° C.

### VII. Pharmaceutical Compositions and Routes of Administration of a TwHF Extract

The disclosure contemplates compositions comprising the freely flowable solid produced by the methods described herein that are tabletted, encapsulated or otherwise formulated for oral administration. The compositions may be provided as pharmaceutical compositions, nutraceutical compositions (e.g., a dietary supplement), or as a food or beverage additive, as defined by the U.S. Food and Drug Administration. The dosage form for the above compositions are not particularly restricted. For example, suspensions, emulsions, tablets, pills, capsules, sustained release formulations, powders, suppositories, liposomes, microparticles, microcapsules and the like are all contemplated as suitable dosage forms.

The compositions typically include one or more suitable diluents, fillers, salts, disintegrants, binders, lubricants, glidants, wetting agents, controlled release matrices, colorants, flavorings, carriers, excipients, buffers, stabilizers, solubilizers, commercial adjuvants, and/or other additives known in the art.

Any pharmaceutically acceptable (i.e., sterile and acceptably non-toxic as known in the art) liquid, semisolid, or solid diluent that serves as a pharmaceutical vehicle, excipient, or medium can be used. Exemplary diluents include, but are not limited to, polyoxyethylene sorbitan monolaurate, magnesium stearate, calcium phosphate, mineral oil, cocoa butter, and oil of theobroma, methyl- and propylhydroxybenzoate, talc, alginates, carbohydrates, especially mannitol, α-lactose, anhydrous lactose, cellulose, sucrose, dextrose, sorbitol, modified dextrans, gum acacia, and starch. Such compositions may influence the physical state, stability, rate of in vivo release, and rate of in vivo clearance of the functional compounds.

Pharmaceutically acceptable fillers can include, for example, lactose, microcrystalline cellulose, dicalcium phosphate, tricalcium phosphate, calcium sulfate, dextrose, mannitol, and/or sucrose. Salts, including calcium triphosphate, magnesium carbonate, and sodium chloride, may also be used as fillers in the pharmaceutical compositions.

Binders may be used to hold the composition containing the freely flowable solid to form a hard tablet. Exemplary binders include materials from organic products such as acacia, tragacanth, starch and gelatin. Other suitable binders include methyl cellulose (MC), ethyl cellulose (EC) and carboxymethyl cellulose (CMC).

The amount and administration regimen of the processed TwHF extract is based on various factors relevant to the purpose of administration, for example human or animal age, sex, body weight, hormone levels, or nutritional need of the human or animal. In some embodiments, the TwHF extract is administered daily to an animal in an amount from about 0.001 mg/kg body weight to about 10 g/kg body weight. In some embodiments, the processed TwHF extract is administered to an animal in an amount of about 0.005 mg/kg body weight per day, or about 0.01, or about 0.05, or about 0.1, or about 0.2, or about 0.3, or about 0.4, or about 0.5, or about 0.6, or about 0.7, or about 0.8, or about 0.9, or about 1, or about 2, or about 3, or about 4, or about 5, or about 6, or about 7, or about 8, or about 9 or about 10 mg/kg body weight per day

A typical regimen may comprise multiple doses of the processed TwHF extract. In one embodiment, the processed TwHF extract is administered once per day. In other embodiments, the processed TwHF extract is administered two or three times per day. The processed TwHF extract may be administered to a subject at any time. In some embodiments, the processed TwHF extract is administered concurrently, prior to or after the consumption of a meal.

Treatment with a processed TwHF extract is continued for at least about 1 week. Duration of treatment lasting about 2 weeks, 3 weeks, 1 month, 2 months, 3 months, 4 months, 5 months, 6 months, 7 months, 8 months, 9 months, 10 months, 11 months, 1 year, 5 years or up to lifetime treatment is also contemplated.

It will be appreciated by one of ordinary skill in the art that the processed TwHF extract described herein is useful in the fields of human medicine and veterinary medicine to provide, for example, triptolide or tripdiolide (or other therapeutic compounds obtainable from debarked roots of a TwHF plant) to a subject in need thereof. Thus, the subject or individual to be treated may be a mammal, such as a human. Other suitable subjects include, for example, farm animals such as cows, sheep, pigs, horses, and goats; companion animals such as dogs and cats; exotic and/or zoo animals; laboratory animals including mice, rats, rabbits, guinea pigs, and hamsters; and poultry such as chickens, turkeys, ducks, and geese.

The following Examples are provided to describe the invention in greater detail, and are intended to illustrate, not to limit, the appended claims. Example 1 describes an exemplary method of freeze-drying and both an exemplary method and an exemplary apparatus for subsequent removal of the bark from the roots of a TwHF plant. Example 2 describes an alternative method of freeze-drying roots of a TwHF plant. Example 3 describes the preparation of a freely flowable solid form of an extract form an initial fluid-based extract of a TwHF plant using hexane as the processing agent. Example 4 describes the preparation of a freely flowable solid from an extract of a TwHF plant using heptane as the processing agent. Example 5 describes an alternative method of preparing a freely flowable solid from an ethanol extract of a TwHF plant using heptane as the processing agent. Example 6 describes the large-scale preparation of a freely flowable solid from an ethanol extract of a TwHF plant using heptane as the co-processing agent. Example 7 describes a method of preparing a freely flowable solid from an extract of TwHF using a formulation excipient as the processing agent. Example 8 discloses an alternative method of producing a freely flowable solid from an ethanol extract of a TwHF plant using microcrystalline cellulose as the processing agent. Example 9 discloses a pharmaceutical composition comprising the freely flowable solid prepared in Example 5.

### EXAMPLES

### Example 1

### Preparation of a Freeze-Dried Debarked Root of a TwHF Plant

The improved TwHF extracts according to the disclosure are more amenable to manipulation as a result of improved flow properties and exhibit improved safety profiles as a result of reduced toxin contamination from T. wilfordii bark components. The reduction in toxin content is achieved by preparing T. wilfordii roots and root portions in a manner that facilitates quantitative or near-quantitative bark removal in an efficient manner. More particularly, the TwHF roots and/or root portions are freeze-dried in preparation for debarking.

A total of 308 g of fresh TwHF root material (91 g medium roots (3-10 mm in length), 15 g small (1.00-3.00 mm in length), 29 g root hairs (1.00 or less in length), 30 g stumps and 143 g large roots (10.00 mm or larger in length)) were placed in Pyrex trays and frozen overnight at −20° C. The freeze-drying (Labconco) chamber was adjusted to −50° C. and allowed to equalize overnight. The root material was then placed in the freeze-drying chamber and the vacuum was set to a pressure of 3-10 millibar. The chamber temperature was raised to 10° C. for four hours, then raised to 25° C. for an additional four hours, and then finally raised to 35° C. for overnight. The resulting freeze-dried root material was removed from the freeze-drying chamber.

Freeze-drying the root material caused cracking of the bark from the root core, which was observed upon visual inspection of the freeze-dried root material. To remove the bark, the freeze-dried root material was pressed or rolled with mild pressure on a flat surface or in a compression device (e.g., a pasta roller) to remove the bark from the root core. The process of removing the bark created a fine beige to pink dust/powder, which was attributed to the bark. Seventeen grams of dried debarked root material were obtained.

The mild pressure did not pulverize the TwHF roots and/or root portions, and these materials were readily separable from any residual dust or powder, ensuring minimal bark toxins would be found in any subsequent root extract. It will be apparent to one of skill in the art that many other mechanical and electromechanical operations will prove suitable for removing the bark from the freeze-dried roots and/or root portions, and all such operations known in the art are contemplated by the disclosure.

The foregoing Example demonstrates that any known method of freezing TwHF root portions known in the art is suitable for use in the methods described herein. Additionally, the freeze-drying process may involve any apparatus capable of controlling the pressure of the atmosphere immediately surrounding the TwHF root portions. As with most freeze-drying methodologies, moreover, the length of time that the initially frozen TwHF root portions are exposed to the freeze-drying process may vary, provided that sufficient time is allowed to reduce the water content of the root portions to acceptable levels, e.g., less than 2%.

### Example 2

### An Alternative Method of Preparing a Freeze-Dried Roots of the TwHF Plant

Consistent with the foregoing discussion and illustrative thereof, an alternative method of freeze-drying TwHF roots and/or root portions involves the use of a heated shelf upon which the root portions have been located. As those of skill in the freeze-drying art would appreciate, the freeze-drying of materials having dimensions sufficient to have water located a distance from any free surface of the material can benefit from relatively mild heating of the material to be freeze-dried (i.e., TwHF roots and/or root portions), without undermining the ability to effectively freeze-dry the material. Without wishing to be bound by theory, it is believed that the mild heating promotes migration of the water from the interior of the substance to a surface where sublimation can, and does, take place.

This Example describes one such alternative method of producing a freeze-dried root of a TwHF plant. Briefly, the temperature of the freeze-drying chamber (Labconco) was set to −20° C. and allowed to equalize overnight. ˜1000 g fresh root material (˜30 cm in length and ˜1-10 mm in diameter) was placed in a container and frozen overnight in a conventional freezer (Frigidaire) until the temperature of the roots was approximately −20° C. The next day, the container comprising the frozen roots was placed in a freeze-drying chamber upon a heated shelf and the pressure of the freeze-drying chamber was set to 3-10 millibars. The temperature of the heated shelf was increased to 10° C. overnight, increased to 35° C. for 8 hours, and then increased to 50° C. for another 8 hours. The freeze-dried root portions were removed from the freeze-drying chamber. The freeze-dried root portions contained less than 20% water, as determined by loss on drying (LOD).

The data provided in this Example establishes that mild heating of the TwHF root portions during the freeze-drying process is tolerated, resulting in production of freeze-dried TwHF root portions. It is believed that this approach to freeze-drying TwHF root portions provides a quicker, and similarly effective, method for freeze-drying TwHF root portions compared to freeze-drying in the absence of mild heating applied during the freeze-drying process.

### Example 3

### Preparation of a Freely Flowable Solid from a Native Extract of a TwHF Plant Using a Non-Polar Organic Fluid as a Co-Processing Agent

TwHF root portions debarked according to the disclosure provide a desirable substrate for extract preparation because the efficient and thorough debarking minimizes, or eliminates, bark toxins that can contaminate TwHF extracts. In addition to addressing the issue of toxins by providing improved debarking procedures, the disclosure provides processes for improving the flow characteristics of extracts prepared from the debarked TwHF root portions. This Example describes a method of producing a freely flowable solid from an extract of a TwHF plant using a non-polar organic fluid (e.g., hexane) as a co-processing agent.

Briefly, 10 g of the native extract obtained from debarked roots from Chinese TwHF plants (i.e., TwHF plant grown in China) were combined with 100 mL hexane (Fisher Reagent Grade) in a 250 mL beaker. A stainless steel spatula was used to break up the extract into pieces. The hexane/extract mixture was then sonicated for 30 minutes at room temperature. The hexane/extract mixture was then placed in a Büchner funnel, filtered and washed with 100 mL hexane to produce a filtrate (which was yellow/orange in color) comprising the hexane wash flow-through and a retentate (which was brown/red in color) comprising the solids in the hexane/extract mixture. The hexane/extract mixture was filtered until the retentate was dry. The retentate was then subjected to repeated washings with 100 mL hexane until a colorless filtrate was observed. The retentate was dried overnight in a vacuum oven (100 millibar, 40° C.) to remove any residual hexane from the retentate. The dried retentate was in the form a freely flowable solid with a yield of 6.7 g.

To assess the scalability of the method to commercial quantities, the process was also applied to a larger quantity of the native extract. Fifty grams of an ethanol extract obtained from debarked roots of Chinese TwHF plants were combined with 250 mL hexane in a 500 mL beaker. Simple extracts of TwHF, such as the ethanol extract, are viscous, almost tar-like, amorphous masses that are difficult to manipulate. A stainless steel spatula was used to break up the extract into pieces. The hexane/extract mixture was then stirred for 30 minutes at room temperature. The hexane/extract mixture was then placed in a Buchner funnel, filtered and washed with 100 mL hexane to produce a filtrate (which was yellow/orange in color) comprising the hexane wash flow-through and a retentate (which was brown/red in color) comprising the solids in the hexane/extract mixture. The hexane/extract mixture was filtered until the retentate was dry. The retentate was then subjected to repeated washings with 100 mL hexane until a colorless filtrate was observed. The retentate was dried overnight in a vacuum oven (CascadeTek; 100 millibar) at room temperature to remove any residual hexane from the retentate. The dried retentate was in the form of a freely flowable solid with a yield of 35.5 g. The results established that the method would scale to commercially useful quantities.

These results showed that a TwHF extract processed by exposure to a non-polar organic fluid such as hexane, would result in a TwHF extract that was a freely flowable solid amenable to manipulations to formulate the extract in forms suitable for nutritional supplementation or therapeutic treatment, including but not limited to such forms as capsules, tablets, gels, creams, and the like. It is expected that any C4-C10 straight-chain or branched-chain alkane or alkene will be suitable in processing TwHF extracts to yield freely flowable extract solids.

### Example 4

### Preparation of a Freely Flowable Solid from a Native Extract of a TwHF Plant Using Another Non-Polar Organic Fluid as a Co-Processing Agent

Consistent with the expectation, stated in Example 3, that any C4-C10 straight- or branched-chain alkane or alkene would be useful in producing a freely flowable solid form of TwHF extract, this Example describes a method of producing a freely flowable solid from an extract of a TwHF plant using a non-polar organic fluid (e.g., heptane) as a co-processing agent.

Ten grams of a native extract from the root of a Chinese TwHF plant was combined with 100 mL heptane (Fisher Reagent Grade) in a 250 mL beaker. A stainless steel spatula was used to break up the extract into pieces. The heptane/extract mixture was then stirred for about 20-30 minutes at room temperature until the extract had dissolved. The heptane/extract mixture was then placed in a Büchner funnel, filtered and washed with 100 mL heptane to produce a filtrate (which was yellow/light orange in color) comprising the heptane wash flow-through and a retentate comprising the solids in the heptane/extract mixture. The heptane/extract mixture was filtered until the retentate was dry. The retentate was then subjected to repeated washings with 100 mL heptane until a colorless filtrate was observed. The retentate was dried overnight in a vacuum oven (100 millibar; 50° C.) to remove any residual heptane from the retentate. The dried retentate was in the form of a freely flowable solid with a yield of 6.4 g.

The experiment was then scaled up and repeated. The native extract (50 g) obtained from debarked roots of Chinese TwHF plants were combined with 300 mL heptane in a 500 mL beaker. A stainless steel spatula was used to break-up the extract into pieces. The heptane/extract mixture was then stirred for about 20-30 minutes at room temperature until the extract had dissolved. The heptane extract mixture was then placed in a Büchner funnel, filtered and washed with 100 mL heptane to produce a filtrate comprising the heptane wash flow-through (which was yellow/light orange in color) and a retentate comprising the solids in the heptane/extract mixture. The heptane/extract mixture was filtered until the retentate was dry. The retentate was then subjected to repeated washings with 100 mL heptane until a colorless filtrate was observed. The retentate was dried overnight in a vacuum oven (CascadeTek; 100 millibar; 50° C.) to remove any residual heptane from the retentate. The dried retentate was in the form of a freely flowable solid with a yield of 30.0 g, establishing that the method would be suitable for commercially useful quantities.

The freely flowable character of TwHF extracts processed with either hexane (Example 3) or heptane (Example 4) establish that contacting a TwHF extract with a non-polar organic fluid, such as a C4-C10 straight- or branched-chain alkane will produce a freely flowable solid form of the extract, amenable to formulation into nutritional supplements or therapeutics.

### Example 5

### Alternative Method of Producing a Freely Flowable Solid from an Ethanol Extract of the Roots of a TwHF Plant Using Heptane as Co-Processing Agent

This Example describes an alternative method of producing a freely flowable solid from an ethanol extract of a TwHF plant. Freeze-dried debarked roots of a TwHF plant were milled and then combined with 8 volumes of ethanol relative to the volume of the roots and stirred at reflux (78° C.) for 6 hours. The ethanol extract was then filtered to produce a first filtrate and moved to a holding tank. The debarked and milled roots were then combined with an additional 8 volumes of ethanol in a second ethanol extraction and stirred at reflux (78° C.) for 6 hours. The second ethanol extract was filtered to produce a second filtrate. The first and second filtrates were combined and then concentrated under vacuum at room temperature to produce a concentrated filtrate mixture. The resulting concentrated filtrate mixture was approximately 0.9 volumes relative to the original root mass.

The concentrated filtrate mixture was then combined with 1.2 volumes ethyl acetate, relative to original root volume, and concentrated under vacuum with the ethanol removed by distillation. This process was repeated until the solvent exchange reduced the ethanol concentration to less than 15% by weight, as determined by NMR, resulting in an ethyl acetate mixture. The ethyl acetate mixture was combined with 3.3 volumes of heptane relative to the original root volume, to produce a heptane mixture, and the mixture concentrated under vacuum, with the ethyl acetate removed by distillation. This process was repeated until the solvent exchange reduced the ethyl acetate concentration to undetectable levels, as determined by NMR. The heptane mixture was then filtered and the solids were collected. The solids were washed with heptane until the flow-through was colorless. The washed solids were then dried in a tray dryer at about 40° C. for about 8-12 hours.

The dried material was a freely flowable solid. This Example confirms that processing an extract of the TwHF plant with a straight-chain C4-C10 alkane, i.e., heptane, results in a freely flowable solid that is suitable for therapeutic or nutritional formulations. Moreover, the Example establishes that there are alternative methods for exposing a TwHF extract to a non-polar organic fluid to produce the extract in the form of a freely flowable solid.

### Example 6

### Large-Scale Preparation of a Freely Flowable Solid from an Ethanol Extract from the Roots of a TwHF Plant Using Heptane as Co-Processing Agent

The process described in Example 5 was repeated multiple times with debarked root portions from Chinese TwHF plants in amounts of approximately 100 kg (n=3), 250.4 kg (n=2), 250.2 kg (n=3) 211.2 kg (n=1) and 286.6 kg (n=1) to produce a freely flowable solid. The starting root material and resulting freely flowable solid material were analyzed by HPLC to fractionate components of the extract and to determine the concentration of triptolide and tripdiolide in the various samples tested. The results of the experiment are set forth in Table 1.

TABLE 1

| | | | | | | | Total | | |
|---|---|---|---|---|---|---|---|---|---|
| | | | | | | | Triptolide | | |
| | | | | | | | and | Total | |
| | | | Triptolide | | Tripdiolide | | Tripdiolide | Triptolide | |
| | Freely | | (μg/mg | Triptolide | (μg/mg | | (μg/mg | and | |
| | flowable | | freely | (μg/g | freely | Tripdiolide | freely | Tripdiolide | |
| | solid | % | flowable | fresh | flowable | (μg/g fresh | flowable | (μg/g fresh | |
| Batch | (grams) | Extracted | solid) | roots) | solid) | roots) | solid) | roots) | Ratio |
| 100 kg | 1180 | 1.18 | 1.07 | 12.6 | 1.75 | 20.7 | 2.82 | 33.3 | 0.61 |
| 100 kg | 1180 | 1.18 | 1.19 | 14.0 | 1.61 | 19.0 | 2.80 | 33.0 | 0.74 |
| 100 kg | 980 | 0.98 | 1.24 | 12.4 | 1.45 | 14.2 | 2.72 | 26.7 | 0.82 |
| 250.4 kg | 3040 | 1.21 | 1.63 | 19.8 | 1.78 | 21.6 | 3.41 | 41.4 | 0.92 |
| 250.4 kg | 3040 | 1.21 | 1.57 | 19.1 | 1.79 | 21.7 | 3.36 | 40.8 | 0.88 |
| 250.2 kg | 2740 | 1.10 | 1.66 | 18.2 | 2.16 | 23.7 | 3.82 | 41.8 | 0.77 |
| 250.2 kg | 2740 | 1.10 | 1.60 | 17.5 | 2.11 | 23.1 | 3.71 | 40.6 | 0.76 |
| 250.2 kg | 2420 | 0.97 | 1.88 | 18.2 | 2.82 | 27.3 | 4.70 | 45.5 | 0.67 |
| 211.2 kg | 2080 | 0.98 | 1.56 | 15.4 | 2.31 | 22.8 | 3.87 | 38.1 | 0.68 |
| 286.6 kg | 3640 | 1.27 | 1.42 | 18.0 | 1.80 | 22.9 | 3.22 | 40.9 | 0.79 |

The experiment described in Example 6 was also repeated multiple times with debarked root portions from a TwHF plant cultivated for several generations in the U.S. to produce a freely flowable solid. The starting root material and resulting freely flowable solid material were analyzed by HPLC to fractionate components of the extract and to determine the concentration of triptolide, tripdiolide and celastrol in the various samples tested. The results of the experiment are set forth in Table 2.

TABLE 2

| | | | | | | | Ratio of |
|---|---|---|---|---|---|---|---|
| | | | | | | Ratio of | Triptolide |
| Extract in the | | | | | | Triptolide | and |
| form of a | | | | | Triptolide + | to | Tripdiolide |
| freely flowable | Yield | Tripdiolide | Triptolide | Celastrol | Tripdiolide | Tripdiolide | to Celastrol |
| solid (mg) | (wt %) | (μg) | (μg) | (μg) | (T + Td) (μg) | (T/Td) | (T + Td/C) |
| 876 mg | 0.35% | 5410.3 | 4295.6 | 12034.4 | 16330.0 | 0.79 | 1.36 |
| 465 mg | 0.19% | 2696.8 | 2431.8 | 8352.6 | 5128.0 | 0.90 | 0.61 |
| 669 mg | 0.27% | 3984.2 | 3508.7 | 1082.9 | 7492.9 | 0.88 | 6.92 |
| 307 mg | 0.12% | 1790.7 | 1407.0 | 902.0 | 2891.7 | 0.94 | 3.21 |
| 505 mg | 0.20% | 2127.8 | 2037.0 | 1208.0 | 4164.8 | 0.96 | 3.45 |
| 964 | 0.39% | 4000.6 | 4588.6 | 2853.4 | 8589.2 | 1.15 | 3.01 |

In another experiment, the process described in Example 5 was repeated with freeze-dried debarked root portions from Chinese TwHF plants in an amount of approximately 250 kg to produce a freely flowable solid. The resulting freely flowable solid material was analyzed by HPLC to fractionate components of the extract and to determine the combined concentration of triptolide and tripdiolide (T+Td) as well as the concentration of celastrol in the freely flowable solid. Results indicated that the freely flowable solid contained 3.76 μg of triptolide and tripdiolide (T+Td) per mg of freely flowable solid and 2.57 μg celastrol per mg of freely flowable solid, yielding a ratio of (T+Td)/celastrol of 1.46.

In yet another experiment, the concentrations of triptolide and tripdiolide in various extracts of TwHF were also analyzed. The results are set forth in Table 3.

TABLE 3

| | Average triptolide | Average tripdiolide |
|---|---|---|
| | concentration | concentration |
| Sample | (μg/g extract) | (μg/g extract) |
| Native extract (before | Not detectable | Not detectable |
| defatting) | | |
| Native extract (after | 154.74 ± 11.81 | 143.93 ± 7.13 |
| defatting) | | |
| Freely flowable solid after | 1452.18 ± 82.46 | 691.24 ± 60.28 |
| hexane wash | | |
| Freely flowable solid after | 1668.54 ± 88.49 | 847.79 ± 12.19 |
| heptane wash | | |
| Second extract before | 5111.32 ± 198.88 | 1877.04 ± 298.16 |
| defatting (Lot #07099) | | |

Tables 1-3 reveal that the freely flowable solid contains concentrated triptolide and tripdiolide compared to the fresh root samples tested. The concentrated levels of triptolide and tripdiolide present in the freely flowable solid produced by processing a native extract of TwHF with heptane allows for the administration of lower dosages of the freely flowable solid to subjects in need of treatment with triptolide and/or tripdiolide in comparison to an extract of TwHF processed by conventional methods.

In addition, the data in this Example demonstrate that the celastrol content is low in extracts obtained from root portions that were freeze-dried prior to bark removal. Thus, the freeze-drying of TwHF root portions prior to bark removal allows for the preparation of extracts containing not only concentrated levels of bioactives (e.g., triptolide and tripdiolide) but also having a reduction in toxicity (e.g., celastrol content) in comparison to the toxicity of an extract of TwHF processed from root portions that were not freeze-dried prior to being debarked.

### Example 7

### Preparation of a Freely Flowable Solid from a Native Extract of a TwHF Plant Using Various Formulation Excipients as Co-Processing Agents

This Example describes processing a native extract of a TwHF plant with various formulation excipients, including maltodextrin, Aerosil®, corn starch, microcrystalline cellulose (MCC) and Neusilin (magnesium aluminometasilicate), to yield a TwHF extract that is a freely flowable solid and is, thus, amenable to further manipulation in the formation of nutritional supplements and therapeutics.

Seven extract samples (1 g each) from root portions of Chinese TwHF plants were individually combined with 100 mL ethanol in seven 250 mL glass beakers. The extract/ethanol mixture was mixed by hand with a stainless steel spatula until the extract was dissolved. Maltodextrin (5 g), Aerosil® (0.2 g), corn starch (5 g), MCC (5 g), MCC (10 g), Neusilin (5 g) or Neusilin (10 g) was then added to separate extract/ethanol mixtures and mixed by hand with a stainless steel spatula until the excipient had been evenly distributed within the extract/ethanol mixture. The excipient/extract/ethanol mixture was then poured into a heat-resistant glass tray, spread into a uniform layer and dried overnight in a vacuum oven (CascadeTek) at 0.155 atm at 50° C. to remove the ethanol from the excipient/extract/ethanol mixture. The results from the various experiments are set forth in Table 4.

TABLE 4

| | Maltodextrin | Aerosil ® | Corn | MCC | MCC | Neusilin | Neusilin |
|---|---|---|---|---|---|---|---|
| | (1 g) | (0.2 g) | Starch (5 g) | (5 g) | (10 g) | (5 g) | (10 g) |
| Weight of | 4.10 g | 0.34 g | 5.10 g | 5.77 g | 10.62 g | 5.83 g | 10.41 g |
| dried | | | | | | | |
| material | | | | | | | |
| % yield | 68.1% | 33.3% | 85% | 96.1% | 96.5% | 97.1% | 94.6% |
| Appearance | Flowable | Flowable, | Flowable | Flowable | Flowable | Flowable, | Flowable, |
| of dried | solid, yet | but | solid, but | solid | solid | but more | but more |
| material | somewhat | somewhat | somewhat | | | cakey than | cakey than |
| | sticky | sticky | cakey | | | the MCC | the MCC |
| | | | | | | (10 g) | (10 g) |
| | | | | | | processed | processed |
| | | | | | | extract | extract |

Results showed that microcrystalline cellulose (MCC) performed better than any other tested excipient in that it produced a TwHF extract in the form of a non-cakey, flowable solid. Although other excipients are expected to prove suitable for use in preparing freely flowable solid forms of TwHF extracts, further work described herein addressed freely flowable solids produced using MCC. Based on the flowability of extracts processed with MCC, the processing of extracts with MCC was scaled up in a manner analogous to the commercial scale experiments described in Examples 4 and 5 and the triptolide and tripdiolide concentrations in the resulting freely flowable solid were analyzed. The results are set forth in Table 5.

TABLE 5

| | Native | MCC | Yield | Tripdiolide | Triptolide | |
|---|---|---|---|---|---|---|
| Sample | extract (g) | (g) | (g) | (μg) | (μg) | T + Td (μg) |
| | 5.451 | 37.88 | 41.1 | 8304.7 | 9507.7 | 17812.4 |
| 5X MCC | 250 | 1250 | 1479.2 | 323648.96 | 312614.13 | 636263.089 |
| 10X MCC | 250.2 | 2500 | 2710.6 | N/A | N/A | 714459.95 |

Results indicated that processing the extract of TwHF with MCC resulted in the production of a freely flowable solid containing high levels of tripdiolide and triptolide. The high levels of triptolide and tripdiolide present in the freely flowable solid produced by processing a native extract of TwHF with MCC allows for the administration of lower dosages of the freely flowable solid, formulated with greater precision, to subjects in need of treatment with triptolide and/or tripdiolide. It is expected that other excipients will also yield freely flowable solid forms of TwHF extracts that exhibit desirable levels of bioactive compounds found in TwHF extracts, such as triptolide and tripdiolide.

### Example 8

### An Alternative Method of Producing a Freely Flowable Solid from an Ethanol Extract of a TwHF Plant Using Microcrystalline Cellulose as a Co-Processing Agent

Described in this Example is an alternative method of producing a TwHF extract in a freely flowable solid form by exposing an ethanol extract of a TwHF plant to an excipient such as MCC. Freeze-dried debarked root portions of a TwHF plant were milled and then combined with 8 volumes of ethanol (approximately 4 L) relative to the mass of the root portions (approximately 500 g) and stirred at reflux (78° C.) for 6 hours. The ethanol extract was then filtered to produce a first filtrate and moved to a holding tank. The debarked and milled root portions were then combined with an additional 8 volumes of ethanol in a second ethanol extraction and stirred at reflux (78° C.) for 6 hours. The second ethanol extract was filtered to produce a second filtrate. The first and second filtrates were combined and then concentrated under vacuum to produce a concentrated filtrate. The resulting concentrated filtrate was approximately 0.9 volumes relative to the original root volume.

The concentrated filtrate was then combined with 1.2 volumes ethyl acetate, relative to original root mass, and concentrated under vacuum at 50° C. with the ethanol removed by distillation. This process was repeated until solvent exchange reduced the ethanol concentration to 15% or less by weight as determined by NMR, resulting in an ethyl acetate mixture. The ethyl acetate mixture was then combined with an appropriate amount of an excipient (compared to the total solid content in the ethyl acetate mixture) and mixed thoroughly. The ethyl acetate/excipient mixture was then concentrated under full vacuum at a temperature not to exceed 55° C. until a dry solids content of approximately 80% was achieved in the ethyl acetate/excipient mixture. The concentrated mixture was then dried in a vacuum dryer under full vacuum at a temperature not to exceed 55° C. to remove ethyl acetate from the concentrated mixture. After the drying process was completed, the dried solid was in the form of a freely flowable solid.

The experiment was repeated using 5× or 10× starch, MCC, maltodextrin, and Neusilin as the formulation excipients and the resulting solids were analyzed to determine the concentration of triptolide and tripdiolide in the various samples. Two of the samples (one 5× sample and one 10× sample) comprising starch as the co-processing excipient received a heptane wash prior to drying the solids to improve the flowability of these samples. The results of the analysis are set forth in Table 6.

TABLE 6

| | Mass | | Mass of | % yield of |
|---|---|---|---|---|
| | of | | freely | Triptolide and |
| | Roots | Root | flowable | Tripdiolide |
| Sample | (g) | Source | solid (g) | from roots |
| 5X starch (not washed | 220 | US | 5.96 | 2.93 × 10⁻³% |
| with heptane) | | | | |
| 5X starch (washed with | 136 | US | 2.9 g | 3.15 × 10⁻³% |
| heptane) | | | | |
| 10X starch (not washed | 268 | US | 11.15 | 3.64 × 10⁻³% |
| with heptane) | | | | |
| 10X starch (washed | 147 | US | 5.17 | 3.00 × 10⁻³% |
| with heptane) | | | | |
| 5X MCC (not washed | 121 | US | 3.65 | 4.57 × 10⁻³% |
| with heptane) | | | | |
| 10X MCC (not washed | 121 | US | 5.88 | 4.22 × 10⁻³% |
| with heptane) | | | | |
| 10X MCC (not washed | 468 | US | 22.27 | 4.03 × 10⁻³% |
| with heptane) | | | | |
| 5X maltodextrin (not | 121 | US | 3.34 | 4.15 × 10⁻³% |
| washed with heptane) | | | | |
| 5X Neusilin (not | 121 | US | 3.12 | 2.97 × 10⁻³% |
| washed with heptane) | | | | |
| 10X Neusilin (not | 121 | US | 5.53 | 2.55 × 10⁻³% |
| washed with heptane) | | | | |
| 10X Neusilin (not | 477 | US | 22.73 | 2.24 × 10⁻³% |
| washed with heptane) | | | | |

It was again observed that processing the extract with MCC produced a non-cakey freely flowable solid that contained high levels of triptolide and tripdiolide, but several other co-processing aids also yielded usefully flowable solid forms of the TwHF extract. Accordingly, it is expected that flowable solid forms of TwHF root extracts will result from exposure of TwHF extracts, e.g., ethanol extracts, to an excipient followed by an optional fluid exchange, and, ultimately a drying of the material.

### Example 9

### Pharmaceutical Compositions

The freely flowable solid form of TwHF extract, prepared according to any of the methods disclosed herein, is suitable for pharmaceutical formulation.

A pharmaceutical formulation was prepared at a concentration of 12% freely flowable solid, a 4:1 ratio of Avicel PH 200 to Lactose 14SD and a talc concentration of 5%. Eighteen grams of the freely flowable solid was used in the pharmaceutical composition or formulation. Top/middle/bottom samples were taken from the blend for content uniformity testing. The resulting blend was uniform.

The batch flowed well in the automated capsule-filling machine. Capsule weights, once achieved, stayed relatively consistent throughout the run. Over 200 capsules were produced with a target fill of 166.67 mg of blend in each capsule and with a target dose of 20 mg of the freely flowable solid. The average capsule fill was 228.1 mg gross weight. With an average empty capsule weight for size 2 capsules of 59.0 mg, the average fill of the blend was 169.1 mg for each capsule. These results establish that the freely flowable solid extract of TwHF disclosed herein is amenable to pharmaceutical formulation using conventional technology and conventional methodologies. One of skill in the art would recognize that capsules, and other forms of packaging therapeutics, are suitable for packaging the freely flowable solid forms of TwHF extracts, and one of skill would further understand that the various pharmaceutical packaging forms are suitable for packaging the freely flowable solid forms of TwHF extracts as nutritional supplements and/or therapeutics.

Numerous modifications and variations in the practice of the invention are expected to occur to those of skill in the art upon consideration of the embodiments disclosed herein. Consequently, the only limitations that should be placed upon the scope of the invention are those that appear in the appended claims.

All of the U.S. patents, U.S. patent application publications, U.S. patent applications, foreign patents, foreign patent applications and non-patent publications referred to in this specification and/or listed in the Application Data Sheet, are incorporated herein by reference, in their entireties.

From the foregoing it will be appreciated that, although specific embodiments of the invention have been described herein for purposes of illustration, various modifications may be made without deviating from the spirit and scope of the invention.

1. A method of processing a native extract of a Tripterygium wilfordii Hook F. (TwHF) plant to produce an extract in the form of a freely flowable solid, the method comprising
(a) mixing the native extract with an alcohol to produce an extract/alcohol mixture;
(b) combining the extract/alcohol mixture with an excipient under vacuum at a temperature of no more than 50° C. to produce a concentrated mixture; and
(c) drying the concentrated mixture at a temperature of no more than 55° C. to remove remaining alcohol, thereby producing the extract in the form of a freely flowable solid.

2. The method of claim 1, wherein the native extract is obtained by one or more ethanol extractions from the root of the TwHF plant.

3. The method of claim 1, wherein the native extract is obtained from a freeze-dried debarked root portion of the TwHF plant.

4. The method of claim 1, wherein the excipient is selected from the group consisting of microcrystalline cellulose, maltodextrin, fumed silica, corn starch, and magnesium aluminometasilicate.

5. The method according to claim 4, wherein the excipient is microcrystalline cellulose.

6. The method according to claim 1, wherein the drying step is performed under a vacuum establishing an air pressure of no more than 0.2 atmospheres (atm).

7. A method of processing an native extract of a Tripterygium wilfordii Hook F. (TwHF) plant to produce the extract in the form of a freely flowable solid, the method comprising:
(a) combining the native extract with a non-polar organic solvent to produce a mixture thereof;
(b) filtering the mixture to isolate a solid precipitate; and
(c) drying the solid precipitate, thereby producing the extract in the form of a freely flowable solid.

8. The method of claim 7, wherein the native extract is obtained by one or more ethanol extractions from the root of the TwHF plant.

9. The method of claim 7, wherein the non-polar organic solvent is selected from the group consisting of a straight-chain C₅-C₁₀ alkane and a branched-chain C₅-C₁₀ alkane.

10. The method of claim 9, wherein the non-organic polar solvent is heptane.

11. The method of claim 7, wherein the native extract is obtained from a freeze-dried debarked root portion of a TwHF plant.

12. An extract of a TwHF plant in the form of a freely flowable solid produced by the method of claim 1.

13. The extract of claim 12, wherein the extract comprises no more than 2.5 μg of celastrol per mg of extract.

14. The extract of claim 12, wherein the extract has a ratio of (triptolide+tripdiolide)/celastrol (T+Td)/C of at least 1.46.

15. The extract of claim 12, wherein the extract has a ratio of (T+Td)/C of at least 1.36.

16. An extract of a TwHF plant in the form of a freely flowable solid produced by the method of claim 7.

17. The extract of claim 16, wherein the extract comprises no more than 2.5 μg of celastrol per mg of extract.

18. The extract of claim 16, wherein the extract has a ratio of (triptolide+tripdiolide)/celastrol (T+Td)/C of about 1.46.

19. The extract of claim 16, wherein the extract has a ratio of (T+Td)/C of About 1.36.

20. A method of processing an alcoholic extract of a Tripterygium wilfordii Hook F. (TwHF) plant to produce an extract in the form of a freely flowable solid comprising:
(a) obtaining an alcoholic extract of a TwHF plant;
(b) exchanging the alcohol in the alcoholic extract for a polar organic solvent to form an extract/polar organic solvent mixture;
(c) combining the extract/polar organic solvent mixture with an excipient under vacuum at a temperature of no more than 50° C. to produce a concentrated mixture; and
(d) drying the concentrated mixture at a temperature of no more than 55° C., thereby producing the alcoholic extract in the form of a freely flowable solid.

21. The method of claim 20, wherein the polar organic solvent is ethyl acetate.

22. A method of processing an alcoholic extract of a Tripterygium Wilfordii Hook F. (TwHF) plant to produce the extract in the form of a freely flowable solid comprising:
(a) obtaining an alcoholic extract of a TwHF plant;
(b) exchanging the alcohol in the alcoholic extract for a polar organic solvent to form an extract/polar organic solvent mixture;
(c) combining the extract/polar organic solvent mixture with a non-polar organic solvent to produce an extract/non-polar organic solvent mixture;
(d) filtering the extract/non-polar organic solvent mixture to isolate a solid precipitate; and
(e) drying the solid precipitate, thereby producing the alcoholic extract in the form of a freely flowable solid.

23. The method of claim 22, wherein the non-polar organic solvent is heptane.
